(19) United States
(12) Plant Patent Application Publication
(10) Pub. No.: US 2012/0331598 P1
(43) Pub. Date: Dec. 27, 2012

(54) Grapevine 'IFG Four'

(50) ;

(75) Inventor: David Cain, Bakersfield, CA (US)

(73) Assignee: International Fruit Genetics, LLC, Bakersfield, CA (US)

(21) Appl. No.: 13/134,949

(22) Filed: Jun. 22, 2011

(51) Int. Cl. A01H 5/00

(52) U.S. Cl. PLT/205

(57) ABSTRACT

A new and distinct grapevine variety denominated ‘IFG Four’ is characterized by producing large, very crisp, dark red, uniform berries with high sugar content borne on medium to large size clusters. The fruit ripen and are commercially harvestable from mid to late August.

### LATIN NAME OF THE GENUS AND SPECIES CLAIMED

Vitis vinifera

### VARIETY DENOMINATION

‘IFG Four’

### BACKGROUND OF THE INVENTION

The new and distinct grapevine described and claimed herein originated from a hand pollinated cross of the Autumn Royal variety (non-patented) and the Crimson variety (non-patented) hybridized in May 2001. The abortive seed traces were subsequently embryo cultured and the resulting plant was planted in the field in April 2002. The present variety of grapevine was selected as a single plant in July 2003 and was first asexually propagated by hardwood cuttings in December 2003. The resulting propagules were planted during April 2004 near Delano, Kern County, Calif. and were found to reproduce true-to-type through at least three generations of asexual reproduction.

### BRIEF SUMMARY OF THE INVENTION

The new grapevine ‘IFG Four’ is characterized by producing naturally large, extremely crisp, elongated dark red seedless berries that require little or no exogenous application of Gibberellic acid to obtain commercially acceptable berry size which ripen in mid-season.

To the inventor's knowledge, the known variety to which the new grapevine variety is most similar is the Scarlet Royal (U.S. Plant Pat. No. 16,229). It can be distinguished from this variety based on unique combination of characteristics, which include naturally larger, more crisp, very uniform berries. Berries of IFG Four are more elongated than Scarlet Royal. Natural berry weight is slightly larger and is substantially larger with the application of Gibberellic acid. Acidity of ‘IFG Four’ is lower than that of Scarlet Royal at a given sugar level. Productivity of ‘IFG Four’ is somewhat lower than Scarlet Royal. ‘IFG Four’ can further be distinguished based on the characteristics described below.

### BRIEF DESCRIPTION OF THE FIGURE

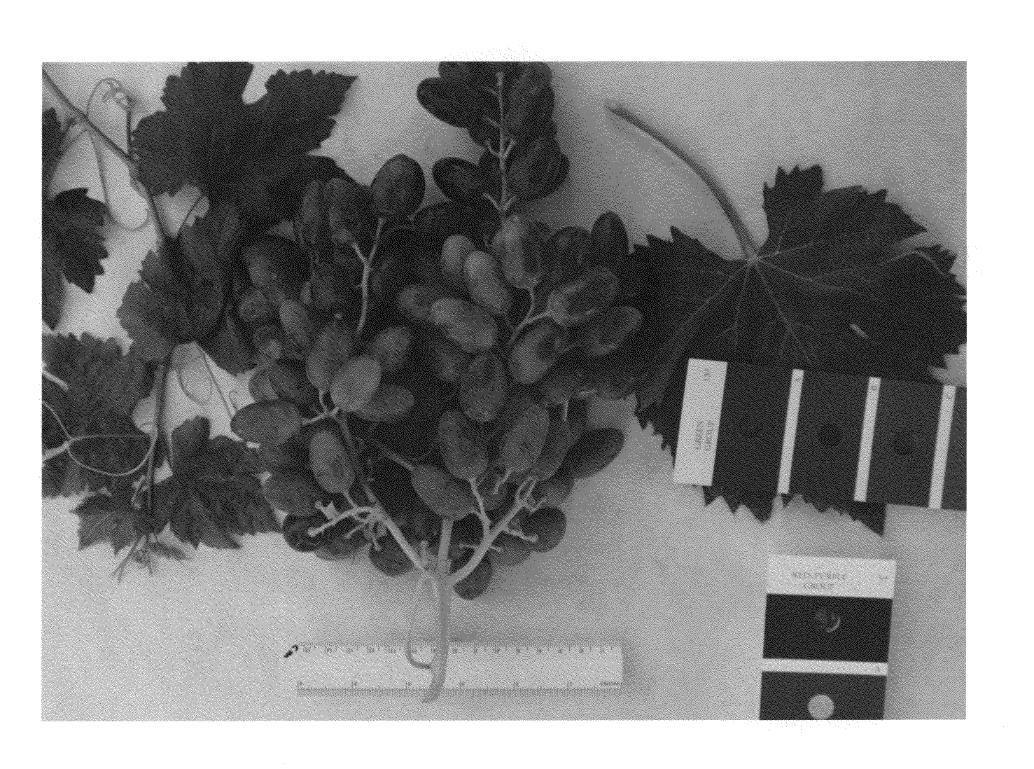

The accompanying photographic illustration in FIG. 1 illustrates in full color ‘IFG Four’. The photograph was taken outdoors with indirect lighting. The colors are as nearly true as is reasonably possible in a color representation of this type.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon R.H.S. Colour Chart, published by The Royal Horticultural Society, London, England.

Throughout this specification subjective description values conform to those set forth by the International Plant Genetic Resources Institute publication ‘Descriptors for Grape’ (vitis spp.) (1983) which was developed in collaboration with the Office International de la Vigne et du Vin (OIV) and the International Union for the Protection of New Varieties of Plants (UPOV).

The descriptive matter which follows pertains to ‘IFG Four’ plants grown in the vicinity of Delano, Kern County, Calif. during 2009 and 2010, and is believed to apply to plants of the variety grown under similar conditions of soil and climate elsewhere:
Vine:
   General.—Size — Large Vigor — Vigorous Density of foliage — Dense Productivity — Medium productive Root stock — Own root Training method — Typically spur pruned leaving 2 bud spurs.
   Trunk.—Trunk diameter of 4-year-old vines at 30 cm above the soil line — 6.7 cm Shape — Stocky Straps — Short — Split Surface texture — Shaggy Inner bark color — Greyed orange; 165A.
Shoots:
   Young shoot.—Form of tip — Wide open Distribution of anthocyanin coloration of tip — Piping (striped) Intensity of anthocyanin coloration of tip — Weak Density of prostrate hairs of tip — Dense Density of erect hairs of tip — Absent Color — 144A, 146B.
   Woody shoot (mature canes).—Shape — Medium Internode length — Medium; About 11.3 cm. Width at node — About 1.3 cm Cross section — Elliptic Surface — Smooth Main color — Yellowish brown; 165B, 174B Density of erect hairs of nodes — None or very sparse Density of erect hairs on internodes — None or very sparse Growth of axillary shoots — Strong; Approximately 30.6 cm.
   Flowering shoot.—Vigor during flowering — Strong Attitude during flowering on shoots not tied — Semi-erect Color — dorsal side of internodes — Green — Green with Red stripes Color — ventral side of internodes — Green Color — dorsal side of nodes — Green with Red stripes Color — ventral side of nodes — Green; 144A Density of prostrate hairs of nodes — Sparse Density of erect hairs of nodes — None Density of prostrate hairs on internode — Very sparse Density of erect hairs on internode — None Anthocyanin coloration of buds — Absent.
   Tendrils.—Distribution on the shoot (at full flowering) — Discontinuous Length of Tendril — Medium — Long; About 20.9 cm Thickness — Medium Color — N144A Form — Bifurcated to Trifurcated Number of consecutive tendrils — 2.
Leaves:
   Young leaves.—Color of upper surface of first four distal unfolded leaves — Copper yellow Average intensity of anthocyanin coloration of six distal leaves prior to flowering — Weak — Medium Density of prostrate hairs between veins (lower surface) — Very sparse Density of prostrate hairs on veins (lower surface) — Medium Density of erect hairs between veins (lower surface) — Absent Density of erect hairs on veins (lower surface) — Sparse.
   Mature leaves.—Average length — About 13.9 cm Average width — About 16.9 cm Mature leaf size — Large Shape of blade — Pentagonal Number of lobes — 5 Anthocyanin coloration of main veins on upper side of blade — Absent Mature leaf profile — Flat Blistering surface of blade upper surface — Weak Leaf blade tip — In the plane of the leaf Undulation of margin — Slight Thickness — Medium Undulation of blade between main and lateral veins — Only near petiole Shape of teeth — Mixture of both sides straight and both sides convex Length of teeth — Medium Ratio length/width of teeth — Equal Shape of upper lateral sinuses — Closed Depth of upper lateral sinuses — Medium General shape petiole sinus — Slightly open to Closed Shape of base of upper leaf sinuses — V-shaped Tooth at petiole sinus — Absent Density of prostrate hairs between veins on lower surface of blade — Very sparse Density of erect hairs between veins on lower surface of blade — Absent Density of prostrate hairs on main veins on lower surface of blade — Sparse — Medium Density of erect hairs on main veins on lower surface of blade — Medium Density of prostrate hairs on main veins on upper surface of blade — Sparse Density of erect hairs on main veins on upper surface of blade — None or very sparse Autumn coloration of leaves — Yellow 11A, Yellow-green 153A, C, D, Grey-purple 183A, B.
   Upper surface.—Color — 137A, B Anthocyanin coloration of main veins — Absent Surface appearance — Semi-glossy Blistering surface of blade — Very weak.
   Lower surface.—Color — 146A, B Anthocyanin coloration of main veins (lower surface) — Absent Glossiness — Medium Surface texture — Smooth Surface appearance — Semi-glossy.
   Petiole.—Length — About 15.4 cm Length of petiole compared to middle vein — Slightly shorter — Equal Density of prostrate hairs on petiole — Sparse Density of erect hairs on petiole — None.
   Buds.—Bud fruitfulness — Basal: Mostly fruitful Position of first fruitful shoot on previous season cane — 2^{nd} to 3^{rd} node Time of bud burst — Late; Mar. 17, 2010.
Flowers:
   General.—Flower sex — Hermaphrodite Length of first inflorescence — Medium; About 13.6 cm long by 8.0 cm wide Position of first flowering and fruiting node — 3^{rd} to 5^{th} node node (current season growth) Number of inflorescence per flowering shoot — 1.1 Time of bloom — Late as compared with similar varieties in the growing area of Delano, Calif. Date of full bloom — May 22, 2010.
Fruit:
   General.—Ripening period — Mid — late; Approximately August 20 in a typical year Use — Fresh market Keeping quality — Medium Resistance to — Insects: Average typical of Vitis vinifera species. Diseases: Average typical of Vitis vinifera specie Shipping quality — Medium Refractometer test — soluble-sugar: About 18.4 Brix Brix/acid — About 47.6 Titratable acidity — About 0.39 Juice pH — About 4.2.
   Cluster.—Mature cluster length (peduncle excluded) — About 32.6 cm Mature cluster width — About 13.7 cm Mature cluster weight — About 802 gm Bunch density — Loose Number of berries — About 165 Form— Cylindrical.
   Peduncle.—Lignification of peduncle — Weak — Medium Length of peduncle — Approximately 3.9 cm.
   Berry.—Uniformity of size — Uniform Single berry weight — About 6.0 g natural; to about 9.1 g when treated with gibberellic acid Shape — Oblong Seeds — Contains small rudimentary seed traces Cross section — Circular Berry dimensions — longitudinal axis: About 26.8 mm horizontal axis: About 18.6 mm Berry firmness — Firm Particular flavor — Neutral Bloom (cuticular wax) — Medium thick Berry separation from pedicel — Medium difficult Skin color (without bloom) — Red-purple; 59A.
   Skin.—Thickness — Thin Texture — Medium tough Reticulation — Absent Tenacity — Tenacious to flesh Tendency to crack — occasional cracks at pedicel.

What is claimed:

1. A new and distinct variety of grapevine as herein illustrated and described.
